(19) United States
(12) Patent Application Publication
(10) Pub. No.: US 2019/0244700 A1
(43) Pub. Date: Aug. 8, 2019

(54) UBERIZATION AND DECENTRALIZATION OF HEALTHCARE SERVICES

(71) Applicant: Aganyan Inc., Van Nuys, CA (US)

(72) Inventors: Hakop Aganyan, Valley Glen, CA (US); Suren Manukyan, Van Nuys, CA (US); Vagarshak Pilossyan, Tarzana, CA (US); Leon K. Kiraj, Glendale, CA (US); Inga Aganyan, Sherman Oaks, CA (US); Silvia Aganyan, Valley Glen, CA (US)

(21) Appl. No.: 15/889,691

(22) Filed: Feb. 6, 2018

(51) Int. Cl. G16H 20/13; G16H 10/60

(52) U.S. Cl. CPC G16H 20/13; G16H 10/60

(57) ABSTRACT

Method and systems for decentralized healthcare services are disclosed. An example system includes a healthcare service center having one or more computer servers and configured to remotely serve patients and healthcare providers. An example method is implemented at least by the healthcare service center and includes the steps of obtaining patient preferences concerning a language or a location of the healthcare providers, maintaining patient profiles, obtaining a request for a healthcare service from a requesting patient, selecting one or more of the healthcare providers using the patient preferences, enabling one or more of the selected healthcare providers to review and respond to the request, establishing a bidirectional communication between the requesting patient and the responding healthcare provider in real-time, and producing a diagnosis of the requesting patient and at least one of a digital diagnostic request, a digital therapy request, and a digital prescription.

### BACKGROUND

### Technical Field

This disclosure generally relates to computer-implemented methods and systems for decentralized healthcare services which involve a healthcare service center enabling at least remote bidirectional communication between patients and healthcare providers, even when they are in different countries.

### Description of Related Art

Today, the cost of healthcare services is constantly growing. Although most Americans purchase health insurance policies to have access to healthcare services at lower prices, there is a large group of people (about 30 million according to some studies) that can't afford paying health insurance premiums or medical bills. As such, the national healthcare system keeps millions of people from even basic medical services. In addition, traditional healthcare services are slow and time-consuming as the current healthcare system facilitates multiple doctor visits to obtain a diagnosis. It often takes a few weeks for a patient from a first visit to obtain a diagnosis and a treatment plan.

In addition, traditional healthcare services, even in emergency rooms, are delaying immediate doctor patient contacts, slow in implementation of planned actions, and time consuming. The current system is not doctor and patient friendly. The current system is not designed to be driven by doctor. Even if the doctor makes a decision to act practically, there are designed obstacles and barriers.

The approaches described in this section could be pursued but are not necessarily approaches that have been previously conceived or pursued. Therefore, unless otherwise indicated herein, the approaches described in this section are not prior art to the claims in this application and are not admitted to be prior art by inclusion in this section.

### SUMMARY

This section is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

The disclosure is concerned with decentralization and uberization of healthcare services. More particularly, the disclosure concerns computer-implemented methods and systems for healthcare services. According to one aspect of this disclosure, there is provided a system for healthcare services. An example system comprises a healthcare service center including one or more computer servers and at least one database. The healthcare service center can be in communication with user devices of patients and user devices of healthcare providers. For these ends, the system also includes a first user interface configured to provide information to the patients and obtain inputs from the patients using a first graphical user interface displayable on the user devices of the patients. The system also includes a second user interface configured to provide information to the healthcare providers and obtain inputs from the healthcare providers using a second graphical user interface displayable on the user devices of the healthcare providers. The healthcare service center is also in communication with at least one diagnostic and laboratory service system configured to perform laboratory or diagnostic operations to the patients and at least one pharmacy service system configured to store and deliver medications to the patients.

According to embodiments disclosed herein, the healthcare service center is configured to: authenticate and authorize the healthcare providers to remotely provide healthcare services to the patients using the healthcare service center, obtain a request for a healthcare service from a requesting patient, selectively establish a bidirectional communication between the requesting patient and a responding healthcare provider in real-time and in accordance with the request for the healthcare service, receive a digital diagnostic request from the responding healthcare provider, cause one of the diagnostic and laboratory service systems to perform a laboratory or diagnostic operation to the requesting patient based on the digital diagnostic request, receive a digital prescription from the responding healthcare provider, and cause one of the pharmacy service systems to provide medication to the requesting patient in accordance with the digital prescription.

According to another aspect of this disclosure, there is provided a method for healthcare services. An example method comprises the steps of: maintaining a healthcare service center that includes one or more computer servers and configured to remotely serve patients and healthcare providers, authorizing (authenticating) the healthcare providers to remotely provide healthcare services to the patients using the healthcare service center, obtaining patient preferences concerning a language or a location of the healthcare providers, maintaining patient profiles in the healthcare service center such that the patients are enabled to remotely configure or modify the patient profiles, obtaining a request for a healthcare service from a requesting patient, selecting one or more of the healthcare providers to be selected healthcare providers using the patient preferences, enabling one or more of the selected healthcare providers to review at least a portion of the request for the healthcare service and respond to the request for the healthcare service, receiving a response to the request for the healthcare service from one of the selected healthcare providers, establishing a bidirectional video teleconference between the requesting patient and the responding healthcare provider in real-time using the healthcare service center, producing a diagnosis of the requesting patient and at least one of a digital diagnostic request, a digital therapy request, and a digital prescription, and updating the patient profile of the requesting patient using the diagnosis.

Additional objects, advantages, novel features, and technical effects of the example embodiments will be set forth in part in the description which follows, and in part will become apparent to those skilled in the art upon examination of the following description and the accompanying drawings or may be learned by production or operation of the example embodiments. The objects and advantages of the concepts may be realized and attained by means of the methodologies, instrumentalities and combinations particularly pointed out in the appended claims. For example, it shall be understood that the technology described in the instant document enables to solve one or more technological problems known in the art, including, for example, the problem of slow, ineffective, and expensive medical data processing and medical data exchange between patients, healthcare service providers, laboratories, pharmacies, and the like.

### BRIEF DESCRIPTION OF THE DRAWINGS

Embodiments are illustrated by way of example and not limitation in the figures of the accompanying drawings, in which like references indicate similar elements and in which:

FIG. 1 shows a high-level block diagram of a system architecture suitable for providing healthcare services according to various embodiments of this disclosure;

FIG. 2 is a process flow diagram showing a method for healthcare services according to an example embodiment;

FIG. 3 is a process flow diagram showing a method for selecting a responding healthcare provider according to an example embodiment; and

FIG. 4 is a block diagram illustrating an example computer system suitable for implementing the methods described herein.

### DETAILED DESCRIPTION OF EXAMPLE EMBODIMENTS

Embodiments of the disclosure provide for decentralized systems and methods that facilitate uberization of healthcare services, including improvements to collection, storage, distribution, visualization, reporting, and analysis of medical data associated with the healthcare services. According to the embodiments of this disclosure, a healthcare service center is provided to connect patients and healthcare providers. The healthcare service center includes one or more computer servers that run a web service accessible by the patients and the healthcare providers via respective user interfaces such as graphical user interfaces available through a website or a software application running on user devices. Both the patients and healthcare providers (e.g., doctors, physicians, dentists, veterinarians, certified healthcare professionals, etc.) can register with the web service and establish patient profiles and healthcare provider profiles, respectively. The healthcare service center may require the healthcare providers to be authenticated and authorized before they can provide medical services via the healthcare service center. The patients can enter their preferences and settings, including, for example, language preferences, residence or location of healthcare providers they are looking for, patients' preexisting medical conditions, recorded or real-time vital parameters, prior diagnostic data, payment and billing data, patient insurance policy data, contact information, an estimated price or quality of healthcare services that the patient seeks, and so forth. The vital parameters can include but are not limited to blood pressure, pulse, oxygen saturation, blood sugar level, and cholesterol levels. The vital parameters can be measured using various in-home devices/sensors (e.g., wearable devices), measured by medical personnel as part of mobile services or self-measured by patients, and transmitted to a platform where they can be accessed by the medical professionals.

Dental x-rays (either partial or full mouth) can be taken by X-ray technicians as part of a mobile service and posted on the platform. Thereafter, medical professionals (e.g., doctors or dentists) selected to provide services can access the vital parameters. The preferences and settings of patients can be stored in patient profiles. Each patient is assigned with a patient identifier, such as a multi-digit access code, which is accessible only to the patient and the healthcare service center.

In operation, a patient starts using the technology described herein when he wants to seek a medical advice or assistance, for example, when the patient has a medical condition (e.g., a flu, abdominal pain, allergy, etc.) or when he wants to undergo a medical examination (e.g., an annual physical examination).

The patient operates a user device such as a personal computer, smart phone, tablet computer, and the like. The user device of the patient runs a browser or a software (mobile) application that the patient can use to access a website associated with a healthcare service center. The patient sends a request for healthcare services to the healthcare service center via the browser or software application. The request can be for a medical consultation, doctor's opinion, medical prescription, and the like. The request for the healthcare services can be associated with a patient profile or patient preferences as to the language or location of prospective healthcare providers. Respectively, the patient may be required to create the patient profile before using the healthcare service center.

The request for the healthcare services may include preliminary data about patient's health or medical condition. In one embodiment, the preliminary data may be as simple as what kind of service the patient is seeking. In other embodiments, the preliminary data can include a description of a medical condition, preexisting condition, photographs, videos, or prior medical records. The patient's request is associated with the patient profile. As such, the healthcare service center links the patient's preferences (e.g., a spoken language) to the patient's request.

The healthcare service center selects a healthcare provider based on the patient's request. The selected healthcare provider is also referred to as a responding healthcare provider. The selection of healthcare provider can vary. In one example, the healthcare service center can process the patient's request and preferences to find the best matching healthcare provider. This process may involve the use of a machine-learning algorithm, heuristic or statistic algorithm.

In another example, the healthcare service center can determine what healthcare providers are currently online and available for service, and then send the patient's request to those healthcare providers. When a group of online healthcare providers receive the patient's request, the healthcare service center selects the healthcare provider who responded first to the patient's request. In this scenario, the healthcare service center can also forward the patient's request to a pre-selected group of healthcare providers based on user preferences. For instance, the healthcare service center can pre-select healthcare providers who speak the language of patient's preference or who are from the country (geographical location) of patient's preference.

Once the responding healthcare provider is selected, the healthcare service center establishes a bidirectional communication between the patient and responding healthcare provider. For example, a video conference can be established between the user device of the patient and a user device of the responding healthcare provider. The user device of the responding healthcare provider can be a smart phone, laptop computer, tablet computer, desktop computer, and the like. In other implementations, the bidirectional communication includes a voice call, texting, chatting, file sharing, and the like. As such, the healthcare service center provides or facilitates decentralized peer-to-peer transactions or consultations between patients and healthcare providers, bypassing the role of centrally planned corporations and insurance companies.

Upon the communication between the patient and the responding healthcare provider, the responding healthcare provider may request diagnostic services, make prescriptions, request outpatient therapy services, and provide a diagnosis for the patient. The responding healthcare provider can also provide referrals to other healthcare providers or therapy centers to perform additional consultations or procedures. The prescriptions, referrals, diagnoses, and requests by the responding healthcare provider are in a digital form and are associated with the patient profile. Further, the medical prescriptions and requests for therapy procedures are automatically sent to respective diagnostic and laboratory service systems and pharmacy service systems.

For example, the diagnostic and laboratory service system can perform, manually or automatically, a diagnostic procedure of the patient based on a diagnostic request provided by the responding healthcare provider. Some example diagnostic operations include, but not limited to, collecting and analyzing fluid samples of the patient (e.g., blood work), radiation service (e.g., x-ray, MRI, or CT scan), or imaging service (e.g., ultrasound scan). In some embodiments, the patient does not even need to leave his home for the diagnostic services. Rather, a mobile diagnostic service vehicle can arrive at the location of the patient to perform the diagnostic operation. For example, a vehicle (including a self-driving vehicle) containing diagnostic equipment can arrive at the patient's location to collect a fluid sample or perform a radiation or imaging scan. The results of diagnostic operations are electronically transmitted to the responding healthcare provider and also stored in the patient profile. The responding healthcare provider may respond by generating a diagnosis and treatment recommendations for the patient. Those can be also stored in the patient profile and delivered to the patient in a digital form. In some embodiments, the healthcare service system can process available patient data (e.g., the patient's request, patient profile data, diagnostic operation results, etc.) to automatically generate a diagnosis or a preliminary diagnosis. For these ends, a machine-learning algorithm can be used.

The responding healthcare provider can also generate a digital prescription for a medication. The digital prescription can be sent automatically to the pharmacy service system. In response thereto, the pharmacy service system can automatically dispatch, ship and deliver the medication to the patient. Similarly, the responding healthcare provider can also generate a request for therapy services for the patient. This request can be electronically delivered to a therapy service provider (e.g., a physical therapy center) to perform the required physical procedures.

Generally, the technology described herein provide for decentralization of healthcare which is a transition to an economic system where medical doctors, patients, and diagnostic personnel can exchange underutilized capacity of existing assets or human resources, while incurring only low transaction costs. The approaches described in this disclosure has different, much lower operating costs compared to traditional business models.

Importantly, the present technology enables patients to select healthcare providers (e.g., medical doctors) who can be located in any country. As discussed above, the patients can communicate with selected healthcare providers using their preferred language. For example, a Russian-speaking patient located in North America can speak with Russian doctor located in Russia through the technology described herein. An Armenian-speaking patient located in North America can speak with an Armenian-speaking doctor in Armenia using this technology. A Mandarin-speaking patient located in North America can communicate and speak with Mandarin-speaking doctor in China using the technology of this disclosure. There are no limitations as to the languages and countries. If a healthcare provider and a patient speak different languages, interpreters can be used.

After consultation with a healthcare provider, and if a diagnosis is established for a patient, the healthcare provider prescribes the necessary medical treatment, medication or diagnostic services (e.g., a blood work). In this case, the healthcare provider contacts a pharmacy service system through the healthcare service center and the patient receives all necessary medications. If the patient needs a detailed diagnostic examination, the healthcare service center refers to a diagnostic center for 80% less than a market price. The diagnostic center is normally serviced by technical personnel who can work with these diagnostic devices anywhere including from home or mobile services. For example, electrocardiogram (EKG or ECG), ultrasound equipment, MRI, X-ray machine, and other lab equipment can be provided in a vehicle, which can arrive to the patient's location for performing required diagnostic services.

The diagnostic center transfers patient diagnostic results data through the healthcare service center to the appropriate healthcare provider located anywhere in the world for reading the patient diagnostic results data and making an accurate diagnosis. In the process of consulting with a healthcare provider and getting information about health, a patient is highly benefited in an hour, regardless of where the patient is located.

The process of diagnosing and providing information (diagnosis, prescriptions, treatment plans, recommendations, diagnostic results) does not last long, and can be done in a matter of hours. At the request of a patient, the data can be sent to another healthcare provider for a second opinion. In addition, the data of each patient, including patient profiles, are stored in the healthcare service center in an encrypted manner and is accessible only to the patient and the patient's selected healthcare providers through the healthcare service center.

Notably, teleconferencing ensures that patients do not infect doctors, medical personnel and other patients. There is no wait time, and the service is much cheaper than traditional medicine routine. Healthcare providers are also available 24 hours a day, seven days a week. It does not matter where a patient is located (at home, work, college, hotel, etc.) to receive qualified medical assistance through the healthcare service center. The patients can receive consultations and individual treatment plans via the healthcare service center. It is also important, there is no need to schedule an appointment. It is also beneficial for people having limited mobility (e.g., disabled or elderly) who can speak with their healthcare providers easily and in real-time. The healthcare providers can be pre-selected for the patients based on language, sex, specialty, affiliation with any hospital, education level and background, rating, ranks, reviews, awards, and the like.

The following detailed description of embodiments includes references to the accompanying drawings, which form a part of the detailed description. Approaches described in this section are not prior art to the claims and are not admitted to be prior art by inclusion in this section. The drawings show illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Aspects of the embodiments will now be presented with reference to a system and methods for providing healthcare services. These system and methods are described in this section and illustrated in the accompanying drawings by various blocks, components, steps, operations, processes, algorithms, and the like, collectively referred to as elements. These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented with a “processing system” that includes one or more processors. Examples of processors include microprocessors, microcontrollers, Central Processing Units (CPUs), digital signal processors (DSPs), field programmable gate arrays (FPGAs), programmable logic devices (PLDs), state machines, gated logic, discrete hardware circuits, and other suitable hardware configured to perform various functions described throughout this disclosure. One or more processors in the processing system may execute software, firmware, or middleware (collectively referred to as “software”). The term “software” shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise. If embodiments of this disclosure are implemented in software, the functions may be stored on or encoded as one or more instructions or code on a non-transitory computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), compact disk ROM (CD-ROM) or other optical disk storage, magnetic disk storage, solid state memory, or any other data storage devices, combinations of the aforementioned types of computer-readable media, or any other medium that can be used to store computer executable code in the form of instructions or data structures that can be accessed by a computer.

For purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

It should be also understood that the terms “first,” “second,” “third,” and so forth can be used herein to describe various elements. These terms are used to distinguish one element from another, but not to imply a required sequence of elements. For example, a first element can be termed a second element, and, similarly, a second element can be termed a first element, without departing from the scope of present teachings.

Moreover, it shall be understood that when an element is referred to as being “on” or “connected” or “coupled” to another element, it can be directly on or connected or coupled to the other element or intervening elements can be present.

The term “user device” shall mean any electronic device with an input and output modules, and also with electronic communication capabilities. The input modules can include a video camera, keypad, touchscreen, trackball, etc. The output modules can include a display, speakers, etc. Some examples of user devices include, but not limited to, a mobile device, cellular phone, mobile phone, smart phone, Internet phone, netbook, tablet computer, laptop computer, desktop computer, personal digital assistant, workstation, thin client, network node, multimedia player, portable computing device, navigation system, in-vehicle computer, smart television device, set top box, game console, entertainment system, infotainment system, and so forth.

The term “patient” shall mean an individual seeking medical service (e.g., a consultation, examination, etc.) and also a user of a user device.

The term “healthcare provider” shall mean a professional who provide healthcare services to patients. Example healthcare providers include a clinician, doctor, physician, specialist (such as a surgeon, radiologist, cardiologist, etc.), dentist, emergency medical technician, physician's assistants, nurse practitioner, nurse, pharmacist, dietician, microbiologist, laboratory expert, laboratory technologist, genetic counselor, researcher, veterinarian, and the like.

Referring now to the drawings, exemplary embodiments are described. The drawings are schematic illustrations of idealized example embodiments. Thus, the example embodiments discussed herein should not be construed as limited to the particular illustrations presented herein, rather these example embodiments can include deviations and differ from the illustrations presented herein.

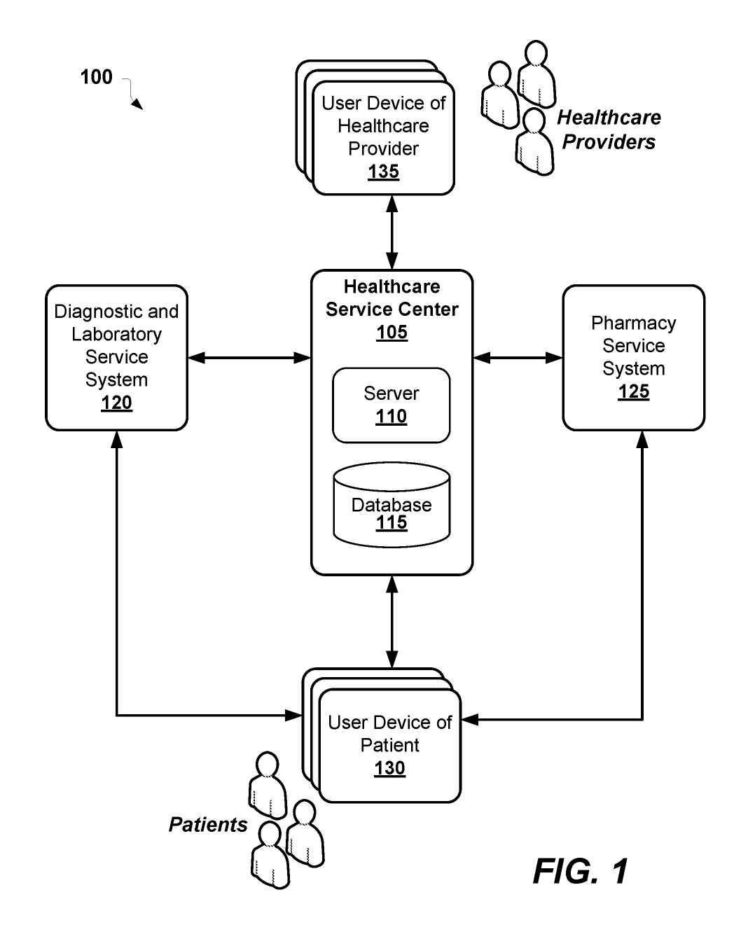

FIG. 1 shows a high-level block diagram of a system architecture 100 suitable for providing healthcare services according to various embodiments of this disclosure. System architecture 100 includes a healthcare service center 105 which includes one or more computer servers 110 and one or more databases 115. It should be noted that the term “server” as used throughout this document refers generally to a computer, a computer program, or a combination thereof. Healthcare service center 105 implements methods for providing healthcare service as described herein by running a software. Healthcare service center 105 can run a web service which can be available to clients (user devices) via Application Programming Interface (API) protocols. In addition, healthcare service center 105 can host a website designed to provide access to the web service and functionality of healthcare service center 105. As such, user devices 130 provide patients with access to a first user interface designed to deliver data to the patients in a visual or audio form, and also obtain data from the patients in the form of text inputs, selections, voice input (which would require natural language processing by healthcare service center 105). Similarly, user devices 135 provide healthcare providers with access to a second user interface designed to deliver data to the healthcare providers in a visual or audio form, and also obtain data from the healthcare providers in the form of text inputs, selections, voice input. Each of the first and second user interfaces could be provided via the website, the software (mobile) application, and the like. Each of the first and second user interfaces could have one or more graphical user interfaces.

System architecture 100 also includes at least one diagnostic and/or laboratory service system 120 and at least one pharmacy service system 125. Diagnostic and laboratory service system 120 refers to an electronic system or facility that performs diagnostic operations to patients. Diagnostic and laboratory service system 120 includes at least one computer or server where patient diagnostic results are collected and stored. Diagnostic and laboratory service system 120 also includes equipment for performing one or more diagnostic or imaging operations such as automated collection and analysis of: patient fluids (e.g., blood, urine, feces, biopsy, etc.), patient images (e.g., x-rays, ultrasound, echo or doppler scans, CT (computed tomography) scans, MRI (magnetic resonance imaging) scans, etc.), patient vital parameters (e.g., a height, weight, temperature, heart rate, oxygen value, blood pressure, bone density, etc.), heart operation (e.g., an electrocardiogram, ECG, EKG), and the like. The equipment may include electronic blood pressure monitoring devices, hematology analyzers, chemistry analyzers, X-ray machines, ultrasound devices, and so forth. Diagnostic and laboratory service system 120 can be at a permanent location or fully mobile. For example, diagnostic and laboratory service system 120 can be built-in a vehicle designed to travel to patient's preferred location. Once diagnostic and laboratory service system 120 automatically obtains patient's fluids, images, or vital parameters (collectively “diagnostic results data”), diagnostic and laboratory service system 120 transfers them to healthcare service center 105 using secure and encrypted methods. The diagnostic results data is associated with respective patient profiles and stored in database 115. The diagnostic results data can be also become available to respective healthcare providers via user interface. In yet additional embodiments, diagnostic and laboratory service system 120 can include accessories, wearable devices, fitness trackers, portable (wearable) medical monitors, and the like. In this implementation, a wearable device (e.g., a smart watch) can perform collection of certain vital parameters (e.g., temperature, heart rate, blood pressure, etc.) and automatically transfer to healthcare service center 105, where this data is associated with the patient profile and also made available to certain healthcare providers.

Pharmacy service system 125 refers to an electronic system or facility that provides medicine (drugs) to the patients. Pharmacy service system 125 includes at least one computer or server where prescriptions associated with patients are received, stored, and processed. Pharmacy service system 125 also includes equipment for storing and dispatching (shipping) the medication for the patients. As such, pharmacy service system 125 can refer to a brick and motor drug store or a large distribution center where medications are automatically selected, transferred, packaged, and shipped to the patients. The delivery can be performed by mail.

Still referring to FIG. 1, system architecture 100 includes user devices 130 that are operated by patients and user devices 135 that are operated by healthcare providers. User devices 130, 135 can be a smart phone or tablet computer enabling the patients and healthcare providers to have bidirectional communication, such as a video or telephone conference. Each user device 130, 135 can include a browser or a software application (e.g., a mobile application) giving access to respective user interfaces.

Healthcare service center 105 is connected to diagnostic and laboratory service system 120, pharmacy service system 125, one or more user devices 130, and one or more user devices 135 via at least one communications network. The communications network refers to any wired, wireless, or optical networks including, for example, the Internet, intranet, local area network (LAN), Personal Area Network (PAN), Wide Area Network (WAN), Virtual Private Network (VPN), cellular phone networks (e.g., Global System for Mobile (GSM) communications network, packet switching communications network, circuit switching communications network), Bluetooth radio, Ethernet network, an IEEE 802.11-based radio frequency network, a Frame Relay network, Internet Protocol (IP) communications network, or any other data communication network utilizing physical layers, link layer capability, or network layer to carry data packets, or any combinations of the above-listed data networks.

Accordingly, system architecture 100 provide for decentralized and automated collection, distribution, and analysis of medical information through user devices. Embodiments facilitate the entry, storage, tracking, visualization, and analysis of comprehensive personal health information.

In additional embodiments, system architecture 100 further includes a payment processing system configured to process online payments of the patients. Healthcare service center 105 can be in operative communication with the payment processing system in order to being able to cause the payment processing system to process a payment of the patients for healthcare services.

In yet other embodiments, healthcare service center 105 can be in communication with an insurance claim system. Healthcare service center 105 is further configured to cause the insurance claim system to provide a payment to the requesting patient or the responding healthcare provider for healthcare services provided by the healthcare provider.

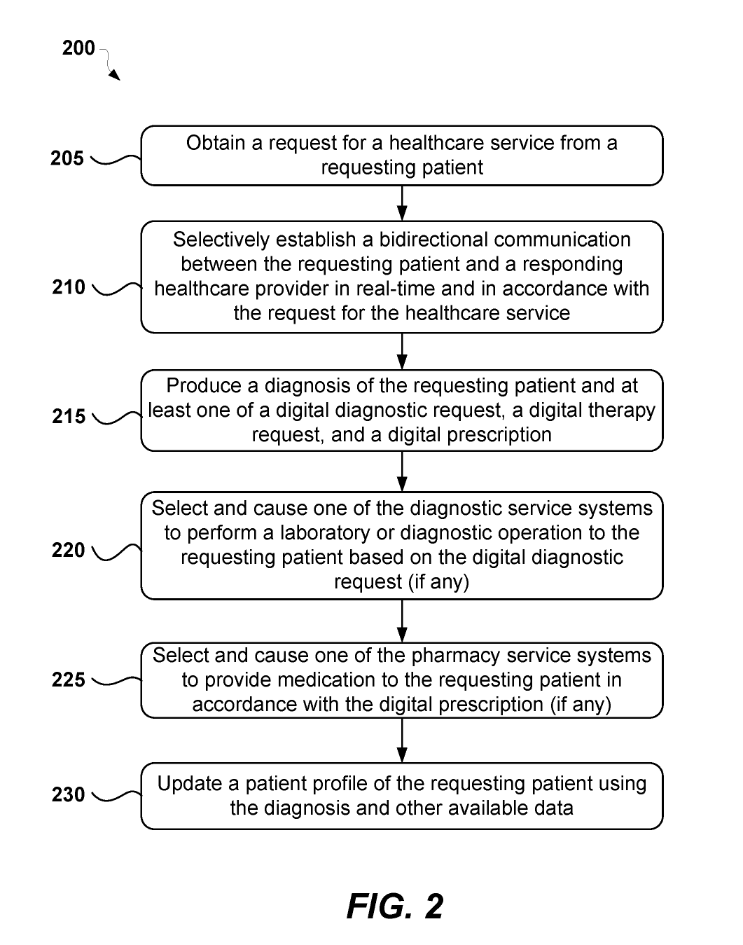

FIG. 2 is a process flow diagram showing a method 200 for (providing) healthcare services according to an example embodiment. Method 200 may be performed by processing logic that may comprise hardware (e.g., decision-making logic, dedicated logic, programmable logic, application-specific integrated circuit (ASIC), and microcode), software (such as software run on a general-purpose computer system or a dedicated machine), or a combination of both. In one example embodiment, the processing logic refers to healthcare service center 105 or any of its components. Below recited operations of method 200 may be implemented in an order different than described and shown in the figure. Moreover, method 200 may have additional operations not shown herein, but which can be evident for those skilled in the art from the present disclosure. Method 200 may also have fewer operations than outlined below and shown in FIG. 2.

Method 200 commences at maintaining healthcare service center 105 that includes one or more computer servers 110 and at least one database 115 as shown in FIG. 1. As discussed above, healthcare service center 105 is configured to provide a web service for one or more patients and one or more healthcare providers. The web service provides a first user interface for serving information to the patients and obtaining inputs from the patients through one or more graphical user interfaces displayable on user devices 130. Similarly, the web service provides a second user interface for serving information to the healthcare providers and obtaining inputs from the healthcare providers using one or more graphical user interfaces displayable on user devices 135 of the healthcare providers. The first and second user interfaces are configured to receive inputs in the form of text, images, selections, touches, taps, voice, video, audio, tactile, haptic, and other inputs, and also configured to deliver outputs in the form of the form of text, images, video, audio, tactile, haptic, and other inputs.

The web service enables the patients to remotely create or modify patient profiles, which can be stored in database 115. The patient profiles can include, for example, a patient's name, address and contact information, payment data (e.g., credit card information), healthcare insurance data, credentials (e.g., a login and password), photographs, images, videos, data concerning medical history, data concerning pre-existing conditions, data concerning health issues, diagnostic reports, medical needs, and the like. The patient profiles can also include one or more patient preferences, including, for example, a patient's spoken language, a preferred language of healthcare provider, a preferred location of healthcare provider, a preferred hospital affiliation of healthcare provider, a preferred specialty of healthcare provider, and the like.

Similarly, the web service enables the healthcare providers to remotely create or modify healthcare provider profiles, which can be stored in database 115. The healthcare provider profiles can include, for example, a name of healthcare provider, address (location) and contact information, specialty (e.g., a family medicine practitioner, cardiologist, ENT, urologist, dermatologist, neurologist, etc.), medical license data, education information, hospital affiliation data, spoken languages, preferred rates, preferred times or schedule of service, banking information, and other necessary personal information.

Before a certain healthcare provider can provide services through healthcare service center 105, the healthcare provider must be authenticated and authorized. As such, in method 200, healthcare service center 105 authenticates and authorizes the healthcare provider to remotely provide healthcare services to the patients using healthcare service center 105. The authentication can involve verifying healthcare provider identity (e.g., through a login and password, two-step verification methods, etc.). The authorization is required to verify that the healthcare provider is duly licensed to provide professional services by a local government. The authorization may involve obtaining medical license data or education information and automatically verifying that the healthcare provider is in a good standing and duly authorized to provide medical services based on the medical license data or education information.

Referring now back to FIG. 2, method 200 continues at block 205 with healthcare service center 105 obtaining a request for a healthcare service from a requesting patient. The request can be input by the patient by a text or voice. If it is a voice input, healthcare service center 105 applies a natural-language processor to transform the voice input into a text input. The patient's input can include one or more healthcare needs or preferences. For example, the request can specify one or more of the following: patient's medical need (e.g., to seek a consultation, to perform a check-up, etc.), a medical condition or concern (e.g., abdominal pain, rash, high temperature, etc.), healthcare provider specialty (e.g., a family doctor, neurologist, gastroenterologist, etc.), preferred language (e.g., English, German, Spanish, French, Russian, etc.), preferred location of healthcare provider (e.g., the United States, Germany, France, Europe, Russia, South Korea, Japan, etc.), and one or more patient's images, videos, audio, and medical data.

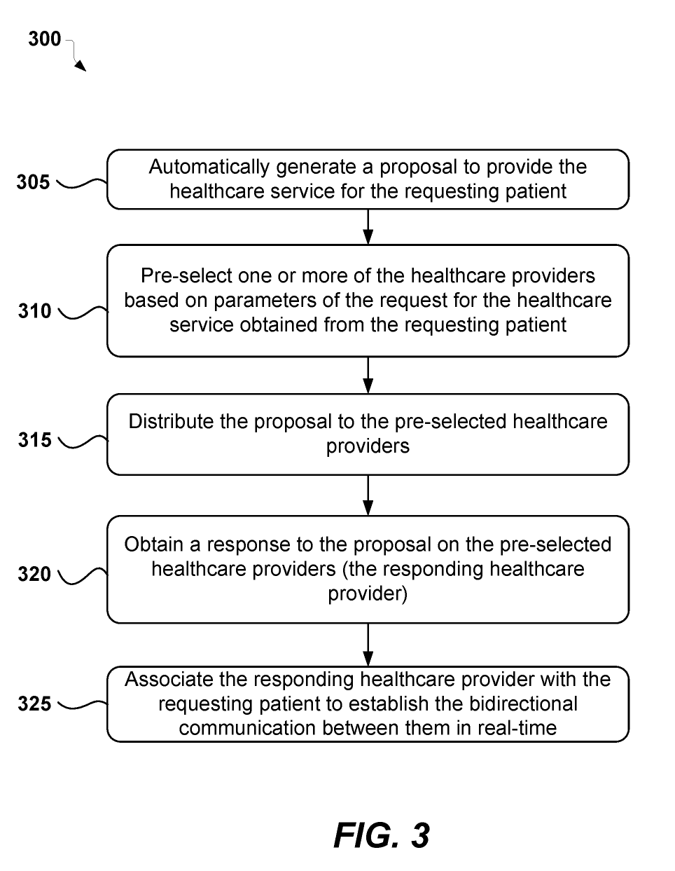

At block 210, healthcare service center 105 selectively establishes a bidirectional communication between the requesting patient and a responding healthcare provider in real-time and in accordance with the request for the healthcare service obtained from the requesting patient at block 205. Healthcare service center 105 can automatically select the responding healthcare provider from a plurality of healthcare providers by matching the patient's preferences or data obtained from the request to the data and preferences of healthcare providers. The matching can be based on scoring and sorting of patient related and healthcare provider related information. In some embodiments, a machine-learning system can be used to perform the matching. In other embodiments, healthcare service center 105 can preselect a certain group of healthcare providers but then enables the preselected healthcare providers to respond in real time to the patient's request. This process explained below and shown in FIG. 3.

The bidirectional communication between the requesting patient and the responding healthcare provider can include one or more real-time data exchange methods such as a video conference, voice call, text chat, file sharing, and the like.

At block 215, healthcare service center 105 automatically produces or receives from the responding healthcare provider a diagnosis of the requesting patient, medical recommendations for the requesting patient, and at least one of a digital diagnostic request, a digital therapy request, and a digital prescription. Any or all of this information can be delivered to the requesting patient through healthcare service center 105 and also stored in the patient profile of the requesting patient. For example, the diagnosis and the diagnostic requests can be entered by the responding healthcare provider via a corresponding user interface. In another example, healthcare service center 105 can automatically recommend and deliver a preliminary diagnosis, any required diagnosis or therapy requests, and prescriptions based on data obtained from the requesting patient or the responding healthcare provider.

In some embodiments, healthcare service center 105 automatically creates one or more recommendations to the requesting patient based on the diagnosis or the digital diagnostic request. Healthcare service center 105 can also transmit the recommendations to the requesting patient.

At optional block 215, healthcare service center 105 selects and causes one of diagnostic and laboratory service systems 120 to perform a laboratory or diagnostic operation to the requesting patient based on the digital diagnostic request (if any). Some example laboratory operations can include collecting and analyzing any body fluids or materials of the requesting patient (e.g., blood, urine, saliva, feces, biopsy sample, etc.). Some example diagnostic operations can include collecting vital parameters (e.g., a heart rate, blood pressure, oxygen level in a blood stream, bone density, body temperature, height, weight) or performing imaging or radiology services (e.g., ultrasound, ECG, MRI, CT, X-ray scans, etc.). Any or all of the laboratory and diagnostic operation can be fully automated.

At optional block 220, healthcare service center 105 selects and causes one of pharmacy service systems 125 to provide medication to the requesting patient in accordance with the digital prescription (if any). Particularly, healthcare service center 105 can select and cause one of pharmacy service systems 125 to receive the digital prescription from the responding healthcare provider and deliver a medication to the requesting patient in accordance with the digital prescription. The delivery operation can include shipping and forwarding the medication by mail.

Similarly, healthcare service center 105 can further receive a digital referral from the responding healthcare provider. The digital referral is associated with the requesting patient. In response to the referral, healthcare service center 105 selects and causes one of physical therapy systems to provide a physical therapy service to the requesting patient in accordance with the digital referral. The physical therapy systems can include automated or manual physical therapy equipment, including, for example, cardiovascular and pulmonary physiotherapy, clinical electrophysiology, geriatric therapy, integumentary therapy, neurological therapy, orthopedic therapy, sports therapy, physiotherapy therapy, and the like.

At optional block 225, healthcare service center 105 updates the patient profile of the requesting patient with the diagnosis, any requests for therapy or diagnosis, medical prescriptions (if any), a summary of medical consultation, one or more health recommendations, and the like.

Additionally, when the requesting patient undergoes a diagnostic procedure as requested by the responding healthcare provider, diagnostic and laboratory service system 120 automatically generates diagnostic reports reflecting collected and analyzed medical data of the requesting patient. Further, diagnostic and laboratory service system 120 transmits the diagnostic report to healthcare service center 105, and healthcare service center 105 stores the diagnostic report in database 115 such that the diagnostic report is associated with the requesting patient in database 115. Further, healthcare service center 105 can automatically determine a preliminary diagnosis of the requesting patient based at least in part on the diagnostic report. Healthcare service center 105 also provides a suggestion message containing the preliminary diagnosis to the requesting patient or the responding healthcare provider. The preliminary diagnosis can also be stored at least temporary in the patient profile. The preliminary diagnosis can be created by using a machine-learning algorithm.

Similarly, the requesting patient can have one more medical equipment suitable to collect vital parameters. For example, the requesting patient can have fitness trackers, electronic blood pressure monitors, wireless scales, medical trackers, and the like, which can automatically collect and transmit the vital parameters to healthcare service center 105 in real time. Healthcare service center 105 stores the real-time vital parameters in database 115 such that they are associated with the requesting patient. Further, healthcare service center 105 automatically determines a preliminary diagnosis of the requesting patient based at least in part on the real-time vital parameters and provides a suggestion message containing the preliminary diagnosis to the requesting patient or the responding healthcare provider.

FIG. 3 is a process flow diagram showing a method 300 for selecting a responding healthcare provider according to an example embodiment. Method 300 can be a part of block 210 discussed above in connection with FIG. 2.

Method 300 may be performed by processing logic that may comprise hardware, software, or a combination of both. In one example embodiment, the processing logic refers to healthcare service center 105 or any of its components. Below recited operations of method 300 may be implemented in an order different than described and shown in the figure. Moreover, method 300 may have additional operations not shown herein, but which can be evident for those skilled in the art from the present disclosure. Method 300 may also have fewer operations than outlined below and shown in FIG. 3.

Method 300 commences at block 305 with healthcare service center 105 automatically generating a proposal to provide a healthcare service for the requesting patient. The proposal can be generated based on the request for the healthcare service obtained from the requesting patient at block 205.

At block 310, healthcare service center 105 pre-selects one or more of the healthcare providers based on parameters of the request for the healthcare service obtained from the requesting patient or based on preferences of the requesting patient stored in the patient profile. The parameters of the request include at least one of requesting patient preferences, the requesting patient preferences include a preferred language of the responding healthcare professional and a residence of the responding healthcare professional. In some embodiments, healthcare service center 105 can pre-select all of the healthcare providers authorized with the web service. In other embodiments, healthcare service center 105 pre-selects only those healthcare providers who are currently online.

At block 315, healthcare service center 105 distributes the proposal to the pre-selected healthcare providers. When received, the proposal can be displayed using the second user interface on a screen of user device 135.

At block 320, healthcare service center 105 obtains a response to the proposal from the responding healthcare provider using the second user interface.

At block 325, healthcare service center 105 associates the responding healthcare provider with the requesting patient to establish the bidirectional communication between the requesting patient and the responding healthcare provider in real-time.

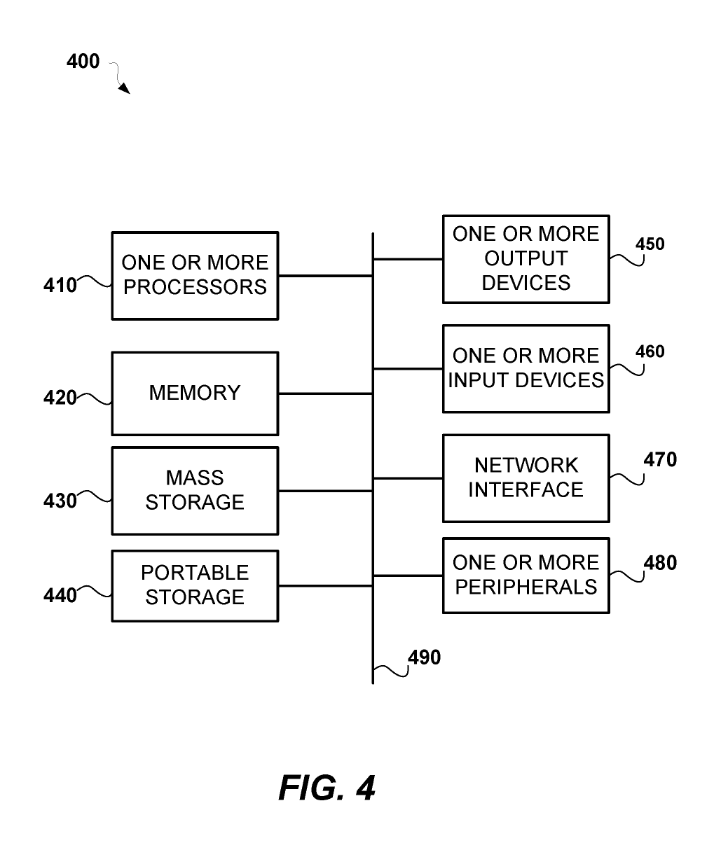

FIG. 4 is a block diagram illustrating an example computer system 400 suitable for implementing the methods described herein. In particular, computer system 400 may be an instance of healthcare service center 105, diagnostic and laboratory service system 120, pharmacy service system 125, user device 130, or user device 135. FIG. 4 illustrates just one example of computer system 400 and in some embodiments computer system 400 may have fewer elements than shown in FIG. 4 or more elements than shown in FIG. 4.

Computer system 400 includes one or more processors 410, a memory 420, one or more storage devices 430, a portable storage 440, one or more input devices 450, one or more output devices 460, network interface 470, and one or more peripherals 480. These elements can be operatively interconnected via a communication bus 490. Processors 410 are, in some examples, configured to implement functionality and/or process instructions for execution within computer system 400. For example, processors 410 may process instructions stored in memory 420 and/or instructions stored on storage devices 430. Such instructions may include components of an operating system or software applications.

Memory 420, according to one example, is configured to store information within computer system 400 during operation. Memory 420, in some example embodiments, may refer to a non-transitory computer-readable storage medium or a computer-readable storage device. In some examples, memory 420 is a temporary memory, meaning that a primary purpose of memory 420 may not be long-term storage. Memory 420 may also refer to a volatile memory, meaning that memory 420 does not maintain stored contents when memory 420 is not receiving power. Examples of volatile memories include random access memories (RAM), dynamic random access memories (DRAM), static random access memories (SRAM), and other forms of volatile memories known in the art. In some examples, memory 420 is used to store program instructions for execution by the processors 410. Memory 420, in one example, is used by software. Generally, software refers to software applications suitable for implementing at least some operations of the methods as described herein.

Storage devices 430 can also include one or more transitory or non-transitory computer-readable storage media and/or computer-readable storage devices. In some embodiments, storage devices 430 may be configured to store greater amounts of information than memory 420. Storage devices 430 may further be configured for long-term storage of information. In some examples, the storage devices 430 include non-volatile storage elements. Examples of such non-volatile storage elements include magnetic hard discs, optical discs, solid-state discs, flash memories, forms of electrically programmable memories (EPROM) or electrically erasable and programmable memories, and other forms of non-volatile memories known in the art.

Still referencing to FIG. 4, computer system 400 may also include one or more input devices 450. Input devices 450 may be configured to receive input from a user through tactile, audio, video, or biometric channels. Examples of input devices 450 may include a keyboard, keypad, mouse, trackball, touchscreen, touchpad, microphone, one or more video cameras, image sensors, fingerprint sensors, or any other device capable of detecting an input from a user or other source and relaying the input to computer system 400 or components thereof.

Output devices 460, in some examples, may be configured to provide output to a user through visual or auditory channels. Output devices 460 may include a video graphics adapter card, a liquid crystal display (LCD) monitor, a light emitting diode (LED) monitor, an organic LED monitor, a sound card, a speaker, a lighting device, a LED, a projector, or any other device capable of generating output that may be intelligible to a user. Output devices 460 may also include a touchscreen, presence-sensitive display, or other input/output capable displays known in the art.

Computer system 400, in some example embodiments, also includes network interface 470. Network interface 470 can be utilized to communicate with external devices via one or more networks such as one or more wired, wireless, or optical networks including, for example, the Internet, intranet, local area network (LAN), wide area network (WAN), cellular phone networks (e.g. Global System for Mobile (GSM) communications network, packet switching communications network, circuit switching communications network), Bluetooth radio, and an IEEE 802.11-based radio frequency network, among others. Network interface 470 may be a network interface card, such as an Ethernet card, an optical transceiver, a radio frequency transceiver, or any other type of device that can send and receive information. Other examples of such network interfaces may include Bluetooth, 3G, 4G, LTE, and Wi-Fi radios in mobile computing devices.

Operating system of computer system 400 may control one or more functionalities of computer system 400 or components thereof. For example, the operating system of computer system 400 may interact with software applications of computer system 400 and may facilitate one or more interactions between the software applications and one or more of processors 410, memory 420, storage devices 430, input devices 450, and output devices 460. Operating system of computer system 400 may interact with the software applications and components thereof. In some embodiments, the software applications may be included in the operating system of computer system 400. In these and other examples, virtual modules, firmware, or software of the software applications. In other examples, virtual modules, firmware, or software may be implemented externally to computer system 400, such as at a network location. In some such instances, computer system 400 may use network interface 470 to access and implement functionalities provided by virtual modules, firmware, or software for vehicle identification through methods commonly known as “cloud computing.”

Thus, methods and systems for healthcare services have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

1. A system for healthcare services, the system comprising:
a healthcare service center including one or more computer servers and at least one database, the healthcare service center being in communication with user devices of patients and user devices of healthcare providers;
a first user interface configured to provide information to the patients and obtain inputs from the patients using a first graphical user interface, the first graphical user interface is displayable on the user devices of the patients;
a second user interface configured to provide information to the healthcare providers and obtain inputs from the healthcare providers using a second graphical user interface, the second graphical user interface is displayable on the user devices of the healthcare providers;
one or more diagnostic and laboratory service systems in communication with the healthcare service center, the one or more diagnostic and laboratory service systems being configured to perform laboratory and diagnostic operations to the patients; and
one or more pharmacy service systems in communication with the healthcare service center, the one or more pharmacy service systems being configured to store medications and deliver the medications to the patients,
wherein the healthcare service center is further configured to:
authenticate the healthcare providers by verifying identity of the healthcare providers so as to enable the healthcare providers to remotely provide healthcare services to the patients using the healthcare service center;
authorize the healthcare providers by verifying their eligibility to provide healthcare services to the patients using the healthcare service center by automatically verifying that each of the healthcare providers is duly licensed to provide professional services by a local government;
obtain a request for a healthcare service from a requesting patient, the requesting patient being one of the patients;
after the request for the healthcare service is obtained, automatically generate a proposal to provide the healthcare service for the requesting patient, the proposal being based on the request for the healthcare service obtained from the requesting patient;
after the proposal to provide the healthcare service is generated, pre-select one or more of the healthcare providers based on parameters of the request for the healthcare service obtained from the requesting patient, the parameters of the request including at least one of requesting patient preferences;
after the one or more of the healthcare providers are pre-selected, distribute the proposal to the pre-selected healthcare providers, the proposal being displayed to each of the pre-selected healthcare providers using the second user interface associated with each of the pre-selected healthcare providers;
after the proposal is distributed, obtain a response to the proposal from one or more of the pre-selected healthcare providers using the second user interface associated with the one or more of the pre-selected healthcare providers;
select, by the healthcare service center, from the one or more of the pre-selected healthcare providers who provided the response to the proposal, a responding healthcare provider, wherein the responding healthcare provider is one of the pre-selected healthcare providers who provided the response to the proposal first;
associate the responding healthcare provider with the requesting patient to establish a bidirectional communication between the requesting patient and the responding healthcare provider in real-time;
selectively establish the bidirectional communication between the requesting patient and the responding healthcare provider in real-time and in accordance with the request for the healthcare service;
after establishing the bidirectional communication, receive a digital diagnostic request from the responding healthcare provider, the digital diagnostic request being associated with the requesting patient;
select and cause one of the one or more diagnostic and laboratory service systems to perform a laboratory or diagnostic operation to the requesting patient based on the digital diagnostic request;
based on the digital diagnostic request, instruct the requesting patient to collect real-time vital parameters of the requesting patient using a vital parameter sensing device associated with the requesting patient and transmit the real-time vital parameters to the healthcare service center;
provide the real-time vital parameters of the requesting patient and results of the laboratory or diagnostic operation to the responding healthcare provider;
receive a digital prescription from the responding healthcare provider, the digital prescription being associated with the requesting patient; and
select and cause one of the one or more pharmacy service systems to provide medication to the requesting patient in accordance with the digital prescription.

2. The system of claim 1, further comprising at least one physical therapy system configured to provide physical therapy operations with the patients, wherein the healthcare service center is further configured to:
receive a digital referral from the responding healthcare provider, the digital referral being associated with the requesting patient; and
select and cause one of the physical therapy systems to provide a physical therapy service to the requesting patient in accordance with the digital referral.

3. (canceled)

4. The system of claim 1, wherein the requesting patient preferences include a preferred language of the responding healthcare provider.

5. (canceled)

6. The system of claim 1, wherein the healthcare service center is configured to pre-select only those ones of the healthcare providers who are currently online.

7. The system of claim 1, wherein the healthcare service center is further configured to:
obtain a diagnostic report in response to the digital diagnostic request from one of the diagnostic and laboratory service systems;
store the diagnostic report in the at least one database of the healthcare service center, the diagnostic report being associated with the requesting patient in the database;
automatically determine a preliminary diagnosis of the requesting patient based at least in part on the diagnostic report; and
provide a suggestion message containing the preliminary diagnosis to the requesting patient or the responding healthcare provider.

8. The system of claim 1, wherein the healthcare service center is further configured to:
obtain real-time vital parameters of the requesting patient;
store the real-time vital parameters in the at least one database of the healthcare service center, the real-time vital parameters being associated with the requesting patient in the database;
automatically determine a preliminary diagnosis of the requesting patient based at least in part on the real-time vital parameters; and
provide a suggestion message containing the preliminary diagnosis to the requesting patient or the responding healthcare provider.

9. The system of claim 1, further comprising a payment processing system configured to process online payments of the requesting patient, wherein the healthcare service center is further in communication with the payment processing system, wherein the healthcare service center is further configured to cause the payment processing system to process a payment of the requesting patient for the healthcare service.

10. The system of claim 1, wherein the healthcare service center is further in communication with an insurance claim system, wherein the healthcare service center is further configured to cause the insurance claim system to provide a payment to the requesting patient or the responding healthcare provider for the healthcare service provided by the responding healthcare provider.

11. A method for healthcare services, the method comprising:
maintaining a healthcare service center including one or more computer servers and at least one database, the healthcare service center being configured to provide a web service for patients and healthcare providers, wherein the healthcare service center is in an operative communication with one or more diagnostic and laboratory service systems configured to perform laboratory or diagnostic operations to the patients and in an operative communication with one or more pharmacy service systems configured to deliver medications to the patients;
authenticating the healthcare providers by verifying identity of the healthcare providers so as to enable the healthcare providers to remotely provide healthcare services to the patients using the healthcare service center;
authorizing the healthcare providers by verifying their eligibility to provide healthcare services to the patients using the healthcare service center by automatically verifying that each of the healthcare providers is duly licensed to provide professional services by a local government;
obtaining a request for a healthcare service from a requesting patient, the requesting patient being one of the patients;
after the request for the healthcare service is obtained, automatically generating a proposal to provide the healthcare service for the requesting patient, the proposal being based on the request for the healthcare service obtained from the requesting patient;
after the proposal to provide the healthcare service is generated, pre-selecting one or more of the healthcare providers based on parameters of the request for the healthcare service obtained from the requesting patient, the parameters of the request including at least one of requesting patient preferences;
after the one or more of the healthcare providers are pre-selected, distributing the proposal to the pre-selected healthcare providers, the proposal being displayed to each of the pre-selected healthcare providers using a second user interface associated with each of the pre-selected healthcare providers;
after the proposal is distributed, obtaining a response to the proposal from one or more of the pre-selected healthcare providers using the second user interface associated with the one or more of the pre-selected healthcare providers;
selecting, by the healthcare service center, from the one or more of the pre-selected healthcare providers who provided the response to the proposal, a responding healthcare provider, wherein the responding healthcare provider is one of the pre-selected healthcare providers who provided the response to the proposal first;
associating the responding healthcare provider with the requesting patient to establish a bidirectional communication between the requesting patient and the responding healthcare provider in real-time;
selectively establishing the bidirectional communication between the requesting patient and the responding healthcare provider in real-time and in accordance with the request for the healthcare service;
after establishing the bidirectional communication, receiving a digital diagnostic request from the responding healthcare provider, the digital diagnostic request being associated with the requesting patient;
selecting and causing one of the one or more diagnostic and laboratory service systems to perform a laboratory or diagnostic operation to the requesting patient based on the digital diagnostic request;
based on the digital diagnostic request, instructing the requesting patient to collect real-time vital parameters of the requesting patient using a vital parameter sensing device associated with the requesting patient and transmit the real-time vital parameters to the healthcare service center;
providing the real-time vital parameters of the requesting patient and results of the laboratory or diagnostic operation to the responding healthcare provider;
receiving a digital prescription from the responding healthcare provider, the digital prescription being associated with the requesting patient; and
selecting and causing one of the one or more pharmacy service systems to provide medication to the requesting patient in accordance with the digital prescription.

12. The method of claim 11, further comprising:
providing a first user interface for serving information to the patients and obtaining inputs from the patients using a first graphical user interface, the first graphical user interface is displayable on user devices of the patients;
providing a second user interface for serving information to the healthcare providers and obtaining inputs from the healthcare providers using a second graphical user interface, the second graphical user interface is displayable on user devices of the healthcare providers.

13. (canceled)

14. The method of claim 11, wherein the requesting patient preferences include a preferred language of the responding healthcare provider.

15. The method of claim 11, further comprising:
obtaining a diagnostic report in response to the digital diagnostic request from one of the diagnostic and laboratory service systems;
storing the diagnostic report in the at least one database of the healthcare service center, the diagnostic report being associated with the requesting patient in the database;
automatically determining a preliminary diagnosis of the requesting patient based at least in part on the diagnostic report; and
providing a suggestion message containing the preliminary diagnosis to the requesting patient or the responding healthcare provider.

16. The method of claim 11, further comprising:
obtaining real-time vital parameters of the requesting patient;
storing the real-time vital parameters in the at least one database of the healthcare service center, the real-time vital parameters being associated with the requesting patient in the database;
automatically determining a preliminary diagnosis of the requesting patient based at least in part on the real-time vital parameters; and
providing a suggestion message containing the preliminary diagnosis to the requesting patient or the responding healthcare provider.

17. A method for healthcare services, the method comprising:
maintaining a healthcare service center including one or more computer servers and at least one database, the healthcare service center being configured to remotely serve patients and healthcare providers, wherein the healthcare service center is in an operative communication with one or more diagnostic and laboratory service systems configured to perform laboratory or diagnostic operations to the patients;
authenticating and authorizing the healthcare providers to remotely provide healthcare services to the patients using the healthcare service center by verifying identity and eligibility of the healthcare providers, wherein the authorizing is performed by automatically verifying that the healthcare providers are duly licensed to provide professional services by a local government;
obtaining patient preferences concerning a language and a country location of the healthcare providers;
maintaining patient profiles of the patients in the at least one database of the healthcare service center, each patient profile including the patient preferences, wherein the healthcare service center is configured to enable the patients to remotely configure or modify the patient profiles;
obtaining a request for a healthcare service from a requesting patient, the requesting patient being one of the patients, wherein the request for the healthcare service is associated with the patient preferences of the requesting patient;
after the request for the healthcare service is obtained, automatically generating a proposal to provide the healthcare service for the requesting patient, the proposal being based on the request for the healthcare service obtained from the requesting patient;
after the proposal to provide the healthcare service is generated, pre-selecting one or more of the healthcare providers based on parameters of the request for the healthcare service obtained from the requesting patient, the parameters of the request including at least one of requesting patient preferences;
after the one or more of the healthcare providers are pre-selected, distributing the proposal to the pre-selected healthcare providers, the proposal being displayed to each of the pre-selected healthcare providers using a second user interface associated with each of the pre-selected healthcare providers;
enabling the pre-selected healthcare providers to review at least a portion of the request for the healthcare service and respond to the request for the healthcare service by generating a response;
after the proposal is distributed, obtaining the response to the proposal from one or more of the pre-selected healthcare providers using the second user interface associated with the one or more of the pre-selected healthcare providers;
selecting, by the healthcare service center, from the one or more of the pre-selected healthcare providers who provided the response to the proposal, a responding healthcare provider, wherein the responding healthcare provider is one of the pre-selected healthcare providers who provided the response to the proposal first;
associating the responding healthcare provider with the requesting patient to establish a bidirectional communication between the requesting patient and the responding healthcare provider in real-time;
after the responding healthcare provider and the requesting patient are associated, establishing the bidirectional video teleconference between the requesting patient and the responding healthcare provider in real-time using the healthcare service center and user interfaces;
after the bidirectional video teleconference is established, producing a diagnosis of the requesting patient and at least one of a digital diagnostic request, a digital therapy request, and a digital prescription;
receiving the digital diagnostic request from the responding healthcare provider, the digital diagnostic request being associated with the requesting patient;
performing a diagnostic operation to the requesting patient based on the digital diagnostic request to obtain a diagnostic report, wherein the diagnostic operation includes at least in part one or more of the following: collecting a fluid sample of the requesting patient, performing an imaging procedure of the requesting patient, and obtaining vital parameters of the requesting patient;
based on the digital diagnostic request, instructing the requesting patient to collect real-time vital parameters of the requesting patient using a vital parameter sensing device associated with the requesting patient and transmit the real-time vital parameters to the healthcare service center;
providing the real-time vital parameters of the requesting patient and results of the laboratory or diagnostic operation to the responding healthcare provider; and
updating a patient profile of the requesting patient using the diagnosis.

18. The method of claim 17, further comprising:
automatically generating the diagnostic report in a digital form based upon an outcome of the diagnostic operation performed to the requesting patient;
transmitting the diagnostic report to the responding healthcare provider using the healthcare service center; and
facilitating the production of the diagnosis of the requesting patient based on the diagnostic report.

19. The method of claim 18, further comprising:
automatically creating one or more recommendations to the requesting patient based on the diagnosis or the digital diagnostic request; and
transmitting the one or more recommendations to the requesting patient using a user interface associated with the healthcare service center.

20. The method of claim 18, further comprising:
receiving a digital prescription from the responding healthcare provider, the digital prescription being associated with the requesting patient; and
delivering from a pharmacy service system a medication to the requesting patient in accordance with the digital prescription.
